(19) United States
(12) Patent Application Publication
(10) Pub. No.: US 2012/0102593 A1
(43) Pub. Date: Apr. 26, 2012

(54) Use of a Histone Deacetylase Gene OsHDT1 in Enhancing Rice Heterosis

(75) Inventors: Daoxiu Zhou, Hubei Province (CN); Chen Li, Hubei Province (CN); Limin Huang, Hubei Province (CN)

(73) Assignee: HUAZHONG AGRICULTURAL UNIVERSITY, Wuhan, Hubei Province (CN)

(21) Appl. No.: 13/266,914

(22) PCT Filed: Apr. 29, 2010
(86) PCT No.: PCT/CN2010/000589

§ 371 Date: Jan. 5, 2012

(30) Foreign Application Priority Data
Apr. 29, 2009 (CN) 200910061829.1

(51) Int. Cl. A01H 5/00; C12N 5/10; A01H 1/06; A01H 5/10

(52) U.S. Cl. 800/278; 800/298; 435/419; 435/412; 435/415; 800/320.1; 800/312; 800/314; 800/320.2

(57) ABSTRACT

A histone deacetylase gene OsHDT1 which enhances utilization of heterosis in rice is isolated and cloned, said gene consisting of: 1) the DNA sequence of positions 1-894 in SEQ ID NO:1 in the Sequence listing; or 2) a DNA sequence which encodes the same protein as that encoded by the DNA sequence of 1). The histone deacetylase gene OsHDT1 is associated with enhanced utilization of heterosis in rice. When the transgenic plants having no phenotype were crossed with “Zhenshan 97A”, the F1 overexpression hybrid plants have obviously earlier flowering period than F1 wild-type hybrid plants. Moreover, the RNAi inhibition hybrid plants have a decreased seed setting rate compared to the negative control hybrid plants, while the parent RNAi inhibition plants and negative control plants shows no evident difference in the trait of seed setting rate. Western blotting revealed that the gene is capable of deacetylating histone, mainly at H4K16 site. In addition, there exist significant differences in the level of histone modification among “Minghui 63”, “Zhenshan 97” and “Shanyou63”.

### TECHNICAL FIELD

The present disclosure pertains to the field of plant genetic engineering. Specifically, the present disclosure relates to isolation, cloning and genetic transformation of a histone deacetylase gene OsHDT1 in order to elucidate, at the level of histone modification, the molecular basis of heterosis.

### BACKGROUND ART

Heterosis, or hybrid vigor, is a phenomenon common in biology that refers to the first filial generation exhibits superior traits to the two parents with different genetic traits (Birchler et al. In search of the molecular basis of heterosis. Plant Cell, 2003, 15: 2236-2239.). Although this phenomenon has been exploited extensively in crop production, research into its mechanism somewhat lags behind. Dominance and overdominance hypotheses are two classical theories regarding the mechanism of heterosis (Hu Jianguang et al., Genetic Basis of Crop Heterosis, Hereditas (Beijing), 1999: 47-50). However, they fail to satisfactorily explain all genetic phenomena. In this case, our laboratory utilized an elite rice hybrid “Shanyou 63” to study effects of genes on yield and its components, and suggested that epistasis (an interaction between nonallelic genes) plays a major role in the formation of heterosis as the important genetic basis of heterosis in Shanyou 63 (Yu et al. Importance of epistasis as the genetic basis of heterosis in an elite rice hybrid. Proc Natl Acad Sci, 1997, 94: 9226-9231). In addition, Professor Bao Wenkui proposed a network system hypothesis in which two different gene clusters from the two parents are combined into a new network system in the first filial generation through hybridization, such that the alleles are in an optimal working state to achieve heterosis (Bao Wenkui, Opportunities and Risks: Reflections on Forty Years of Breeding Practice, Plants, 1990: 4-5). Hua Jinping has, to some degree, provided molecular evidence for gene network system using “Yongjiu F2” rice population (Hua et al. Single-locus heterotic effects and dominance by dominance interactions can adequately explain the genetic basis of heterosis in an elite rice hybrid. Proc Natl Acad Sci, 2003, 100: 2574-2579).

Epigenetics relates to changes in gene expression level caused by alterations other than those of gene sequences, e.g. DNA methylation, histone modification and chromatin remodeling etc. A number of studies from plants reveal the correlation of DNA methylation with regulation of gene expression (Meyer et al., 1994). In recent years, studies on the regulation of DNA methylation and transcription level were performed to elucidate the genetic mechanism of heterosis.

Investigation into the proportions of methylated cytosine in a corn hybrid and its parents revealed a lower degree of methylation in the hybrid than its parents (27.4% for the hybrid compared to 31.4% and 28.3% for its parents), and a significant negative correlation between the activity of gene expression and DNA methylation. Thus, it was suggested that heterosis is achieved by increased gene expression due to hybridization (Tsaftaris A S, and Kafka M. Mechanism of heterosis in crop plants. Journal of Crop Production, 1998, 1: 95-111). However, an opposite result was obtained by study on DNA methylation in a rice hybrid “Shanyou 63” and its parents “Zhenshan 97” and “Minghui 63” (16.3% for both parents but as high as 18% for the hybrid). The overall degree of DNA methylation in the rice hybrid was irrelevant to its heterosis. Nevertheless, increased or decreased methylation on certain specific sites had a marked effect on heterosis. This analysis result could be confirmed by that of the relationship between differential gene expression and heterosis, and vice versa (Xiong et al. Patterns of cytosine methylation in an elite rice hybrid and its parental lines, detected by a methylation-sensitive amplification polymorphism technique. Mol Gen Genet, 1999, 261: 439-446).

It is obvious that heterosis is in fact the consequence of gene regulation. Histone modification, which is an important aspect of epigenetics, as a means of gene regulation, has an important effect on gene transcription, and thus should be connected with heterosis to some extent. Studies with Arabidopsis thaliana in recent years have revealed that some regulatory genes in the hybrid exhibited some alterations at the level of histone acetylation relative to those in the parents. These alterations would influence the expression of downstream genes, which in turn would result in changes in the physiology and metabolic pathways and hence variation in the phenotypes of the plant (Ni et al. Altered circadian rhythms regulate growth vigour in hybrids and allopolyploids. Nature, 2008).

With the elucidation of the molecular mechanism of heterosis, the theoretical basis for heterosis is taking shape. Nevertheless, the mechanism of heterosis at the level of chromatin modification is still unclear. The present inventors explored, at the level of histone modification, the molecular basis of rice heterosis by isolation, cloning and genetic transformation of a histone deacetylase gene OsHDT1. Some altered traits were found and analyzed at molecular level, and the linkage between heterosis and epigenetic phenomena has been preliminarily established.

In searching rice protein sequences in the NCBI database, a histone deacetylase OsHDT1 belonging to rice HD2 family (a histone deacetylase family unique to plants) was found by aligning with Histone deacetylase domain (HDAC Domain). The full-length cDNA for the enzyme has an accession number of AK072845 and a gene ID number of 4339823 (see Table 1). The function of this gene in rice remains unclear.

### DISCLOSURE OF THE INVENTION

It is an object of the present disclosure to isolate and clone a histone deacetylase gene OsHDT1 and use the gene to enhance heterosis in rice and other crop plants.

The object of the present disclosure is achieved by the following technical solution:

The present inventors have isolated from rice a histone deacetylase gene OsHDT1, which heterotically regulates flowering period and seed setting percent of rice. The coding gene of OsHDT1 is as shown in SEQ ID NO:1 in the Sequence Listing. The full-length sequence has 894 bases, encoding 298 amino acids.

OsHDT1 gene was verified to be associated with hetereosis in rice. The complete coding sequence of the gene was linked to a corn ubiquitin promoter and transformed into rice. The transformed rice was crossed with a sterile line “Zhenshan 97A” (obtained from Jiangxi Academy of Agricultural Sciences, Jiangxi, China). The transgenic hybrid plants had an obviously earlier flowering period than wild-type control hybrid plants; while the hybrid plants obtained from the cross of parent plants into which double stranded inhibitory vectors have been introduced showed markedly decreased seed setting rate relative to wild-type control hybrid plants. Through analysis, it was found that OsHDT1 gene regulates the level of acetylation in the parents and hybrid plants via histone modification, resulting in differences in traits among rice varieties.

As shown in FIG. 1, the inventors of the present disclosure constructed an overexpression vector pU1301 and an expression inhibitory vector pDS1301 to obtain transforming vectors pU1301-HDT1 and pDS1301-HDT1. A rice variety “Minghui 63” (a published indica rice subspecies, obtained from Sanming Institute of Agricultural Science, Fujian, China) was transformed with the transforming vectors to obtain transgenic rice plants. The procedure was carried out as follows:
1) primer pairs were designed based on the DNA sequence of the gene obtained from the NCBI database, and used to amplify CDS region;
2) an overexpression vector pU1301 and an expression inhibitory vector pDS1301 were constructed to obtain transforming vectors pU1301-HDT1 and pDS1301-HDT1;
3) the OsHDT1 gene was introduced into a rice recipient using Agrobacterium-mediated transformation method to obtain transformed plants;
4) positive transgenic plants were identified using RT-PCR and Northern blotting, and the transgenic plants were detected for copy number using Southern blotting and observed for phenotypes;
5) single-copy transgenic plants with no phenotypic change were crossed with a sterile line “Zhenshan 97A”, and the hybrid progenies and the self-bred progenies were concurrently planted in field for phenotype observation, agronomic trait assessment and statistical analysis;
6) the expression patterns of rice endogenous OsHDT1 gene in various varieties were analyzed using Northern blotting;
7) the expression levels of the gene in transgenic and wild-type parents as well as hybrid plants were analyzed using RT-PCR;
8) histone modification in the transgenic plants was analyzed using Western blotting.

The invention relates to use of a histone deacetylase gene OsHDT1 for enhancing utilization of rice heterosis. Said gene may be from rice and preferably consists of:
1) the DNA sequence of positions 1-894 in SEQ ID NO: 1 in the Sequence Listing; or
2) a DNA sequence which encodes the same protein as that encoded by the DNA sequence of 1).

This invention also provides transgenic plant cells comprising the stably integrated recombinant DNA constructs of the invention, transgenic plants and seeds comprising a plurality of such transgenic plant cells and transgenic pollen of such plants. Such transgenic plants are selected from a population of transgenic plants regenerated from plant cells transformed with recombinant DNA constructs by screening transgenic plants for an enhanced trait as compared to control plants. The enhanced trait is one or more of enhanced water use efficiency, increased yield, and enhanced nitrogen use efficiency.

As used herein, an “enhanced trait” means a characteristic of a transgenic plant that includes, but is not limited to, an enhance agronomic trait characterized by enhanced plant morphology, physiology, growth and development, yield, nutritional enhancement, disease or pest resistance, or environmental or chemical tolerance. In more specific aspects of this invention enhanced trait is selected from group of enhanced traits consisting of enhanced water use efficiency, increased yield, and enhanced nitrogen use efficiency. In an important aspect of the invention the enhanced trait is enhanced yield including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density.

“Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), panicle number, seed number per ear, seed size, composition of seed (starch, oil, protein) and characteristics of seed fill.

Increased yield of a transgenic plant of the present invention can be measured in a number of ways, including test weight, seed number per plant, seed weight, seed number per unit area (i.e., seeds, or weight of seeds, per acre), bushels per acre, tons per acre, or kilo per hectare. For example, corn yield may be measured as production of shelled corn kernels per unit of production area, for example in bushels per acre or metric tons per hectare, often reported on a moisture adjusted basis, for example at 15.5 percent moisture. Increased yield can be expressed in actual delta bushels per acre compared to control or % change of bushels per acre compared to control. Increased yield may result from improved utilization of key biochemical compounds, such as nitrogen, phosphorous and carbohydrate, or from improved responses to environmental stresses, such as cold, heat, drought, salt, and attack by pests or pathogens. Recombinant DNA used in this invention can also be used to provide plants having improved growth and development, and ultimately increased yield, as the result of modified expression of plant growth regulators or modification of cell cycle or photosynthesis pathways.

Although the plant cells and methods of this invention can be applied to any plant cell, plant, seed or pollen, e.g., any fruit, vegetable, grass, tree or ornamental plant, the various aspects of the invention are preferably applied to corn, soybean, cotton, canola, alfalfa, wheat, rice, sugarcane, and sugar beet plants.

In an embodiment, this invention provides a transformed plant comprising a recombinant DNA construct comprising a promoter functional in a plant cell positioned to provide for expression of a polynucleotide having a sequence with at least about 70%, 75%, 80%, 85%, 90%, or 95% identity to SEQ ID NO:1.

In another embodiment, this invention provides a transformed plant comprising a recombinant DNA construct comprising a promoter functional in a plant cell positioned to provide for expression of a polynucleotide encoding a polypeptide with at least about 70%, 75%, 80%, 85%, 90%, or 95% identity to SEQ ID NO:2.

In a further embodiment, this invention provides a method of producing a transformed plant having an improved property, wherein said method comprises transforming a plant with a recombinant construct comprising a promoter functional in a plant cell positioned to provide for expression of a polynucleotide having a sequence with at least about 70%, 75%, 80%, 85%, 90%, or 95% identity to SEQ ID NO:1, and wherein said improved property is selected from the group consisting of enhanced water use efficiency, increased yield, enhanced nitrogen use efficiency, altered flowering time, and altered seed setting, or combinations thereof.

In an embodiment, this invention provides a method of producing a transformed plant having an improved property, wherein said method comprises transforming a plant with a recombinant construct comprising a promoter functional in a plant cell positioned to provide for expression of a polynucleotide encoding a polypeptide with at least about 70%, 75%, 80%, 85%, 90%, or 95% identity to SEQ ID NO:2, and wherein said improved property is selected from the group consisting of enhanced water use efficiency, increased yield, enhanced nitrogen use efficiency, altered flowering time, and altered seed setting, or combinations thereof.

In another embodiment, this invention provides a transformed plant exhibiting an improved property as compared to the control plant, wherein the altered trait is selected from the group consisting of enhanced water use efficiency, increased yield, enhanced nitrogen use efficiency, altered flowering time, and altered seed setting, or combinations thereof, wherein the plant has greater expression or activity of a polypeptide encoded by a polynucleotide that has at least about 70%, 75%, 80%, 85%, 90%, or 95% identity to SEQ ID NO:1.

In a further embodiment, this invention provides a transformed plant exhibiting an improved property as compared to the control plant, wherein the altered trait is selected from the group consisting of enhanced water use efficiency, increased yield, enhanced nitrogen use efficiency, altered flowering time, and altered seed setting, or combinations thereof, wherein the plant has greater expression or activity of a polypeptide with at least about 70%, 75%, 80%, 85%, 90%, or 95% identity to SEQ ID NO:2.

In an embodiment, this invention provides a seed meal obtained from a seed of the transformed plant as described above.

In the above embodiments, the plant is preferably a crop plant.

In an embodiment, this invention provides a transgenic plant cell comprising a recombinant DNA construct comprising a promoter that is functional in a plant cell and that is operably linked to a polynucleotide that, when expressed in a plant cell encodes a polypeptide with at least about 70%, 75%, 80%, 85%, 90%, or 95% identity to SEQ ID NO:2, wherein said plant cell is selected by screening a population of transgenic plant cells that have been transformed with said construct for altered histone acetylation.

In certain embodiments, said plant cell is part of a transgenic plant. In other embodiments, said plant cell is part of a corn, soybean, cotton, canola, alfalfa, wheat, rice, sugarcane, or sugar beet plant.

A more detailed illustration of the present disclosure is set forth in the following examples. The examples and embodiments described herein are for illustrative purposes only and various modifications or changes suggested to persons skilled in the art are to be included within the purview of disclosure and scope of the appended claims. For examples, in the above embodiments, the histone deacetylase gene OsHDT1 or the protein encoded thereby can be any OsHDT1 gene or its protein from rice rather than SEQ ID NO: 1 or 2, or a homologue from other plants.

### BRIEF DESCRIPTION OF THE DRAWINGS

SEQ ID NO: 1 in the Sequence Listing shows the encoding region of OsHDT1 gene isolated and cloned according to the present disclosure, and the amino acid sequence encoded.

FIG. 1 is a schematic of vectors. A: an overexpression vector PU1301; B: an expression inhibitory vector pDS1301.

FIG. 2 shows the detection of copy number of OsHDT1 gene in T0 transgenic “Minghui 63” plants, indicating that all transgenic lines detected had a single copy except for PR-8 line, which had two copies.

FIG. 3 shows the phenotypes of OsHDT1 transgenic parent and hybrid plants. Panel A shows that the overexpression hybrid plants had obviously earlier heading period. Panel B presents statistical analysis of flowering period of various plants, with asterisk representing significant difference (*P<0.05) or extremely significant difference (**P<0.01) between transgenic lines and wild-type lines. Panel C presents the t-test analysis of flowering period of various plants (A, B and C represent extremely significant difference (P<0.01); and a, b and c represent significant difference (P<0.05)). Panel D shows variance analysis of the trait, seed setting rate, in parent and hybrid transgenic positive plants and negative control plants.

FIG. 4 depicts expression pattern of OsHDT1 gene. Panel A shows the expression pattern of OsHDT1 in various tissues of “Minghui 63”, with higher expression in buds, stems, and phase III and phase V young ears, and lower expression in young leaves and flag leaves. Panel B indicates that there is no marked difference in the expression levels of OsHDT1 in various plants.

FIG. 5 depicts subcellular localization of HDT1, showing that this protein was localized in the nucleus (a, GFP control; b, HDT1-GFP; c, PI staining; d, bright field. Bar=40 μm).

FIG. 6 shows analysis of expression levels of OsHDT1 in transgenic and wild-type parent and hybrid plants.

FIG. 7 shows investigation of the function of HDT1 in histone modification using Western blotting.

FIG. 8 shows rhythmicity of endogenous genes. A: in “Minghui 63”; B: in “Shanyou 63”; LD: long sunlight treatment; SD: short sunlight treatment; MH: “Minghui 63” wild-type; SY: F1 SY63 wild-type obtained by crossing MH with “Zhenshan 97” (ZS97).

FIG. 9 shows analysis of expression of flowering-related genes. A, C, E and G: in “Minghui 63”; B, D, F and H: in “Shanyou 63”; A and B: Hd3a; C and D: Ehd1; E and F: Hd1; G and H: OsGI. LD: long sunlight treatment; SD: short sunlight treatment; MH: “Minghui 63” wild-type; PU: OsHDT1-overexpressing material obtained using MH63 as a recipient. SY: SY63 wild-type; FU: F1 generation material obtained by crossing PU with ZS97.

FIG. 10 shows cluster analysis on chip data of expression profiles of histone acetylase and deacetylase genes in rice. M: “Minghui 63”; S: “Shanyou 63”; Z: “Zhenshan 97”.

### EXAMPLES

### Example 1

### Cloning and Sequence Analysis of OsHDT1 Gene

The histone deacetylase gene OsHDT1 according to the present disclosure, which is a reporter gene (see Table 1 for the details), was amplified using RT-PCR (See: Sambrook, J., E. F. Fritsch, and T. Maniatis, Molecular Cloning: a Laboratory Manual (3^{rd} edition), translated by Huang Peitang, Wang Jiaxi et al., Science Press (China), 2002 edition) to obtain its full-length encoding sequence.

The procedure was carried out as follows. Primers were designed based on the full-length cDNA sequence of rice OsHDT1 gene published in public databases (http://www.ncbi.nih.gov/; http://cdna01.dna.affrc.go.jp/cDNA/) followed by performing PCR amplification. The amplified products were ligated into pGEM T-vector (Promega) by T/A cloning and verified by sequencing. The primers used to clone the full-length gene were FLHDT1-F and FLHDT1-R, with their sequences shown in Table 4 below.

RNAi inhibitory fragments were obtained in the same manner. The primers used to clone RNAi inhibitory fragments were HDT1RNAi-F and HDT1RNAi-R, with their sequences shown in Table 2 below.

TABLE 1
Details about the OsHDT1 gene according to the present disclosure

| | | | | Chromosomal |
|---|---|---|---|---|
| | NCBI | | Full-length | localization |
| Gene name | Accession no. | Gene ID | cDNA | (Locus) |
| OsHDT1 | AF255711 | 4339823 | AK072845 | Os05g0597100 |

### Example 2

### Construction of Dual Ti Plasmid Vector and Establishment of Transformed Agrobacterium

The procedure was carried out as follows:

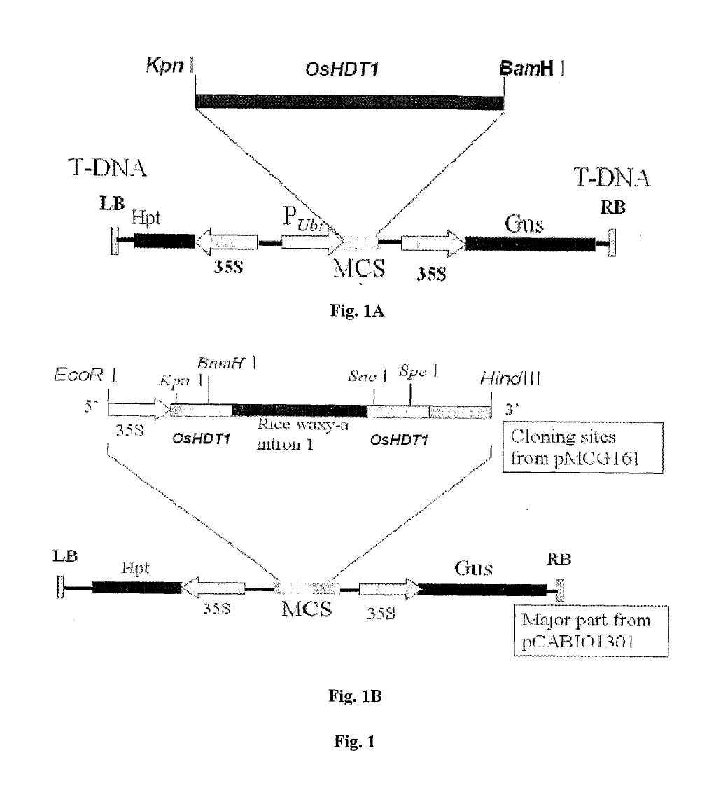

(1) The T/A clone carrying the full-length cDNA of OsHDT1 was digested with KpnI and BamHI. The target fragment was recovered and ligated with expression vector plasmid pU1301 which has also been digested with KpnI and BamHI (FIG. 1A, see Huang et al., Down-regulation of a Silent Information Regulator2-related gene, OsSRT1, induces DNA fragmentation and cell death in rice. Plant Physiol, 2007, 144: 1508-1519) to construct an overexpression vector. The T/A clone carrying the full-length OsHDT1 interfering fragment was digested with KpnI and BamHI. The target fragment was recovered and ligated with expression vector plasmid pDS 1301 which has also been digested with KpnI and BamHI (FIG. 1B, see Chu et al., Promoter mutations of an essential gene for pollen development result in disease resistance in rice. Genes Dev, 2006, 20: 1250-1255) (The endonucleases used were all from TAKARA Co. Ltd, and were used according to manufacturer's instruction; and the ligase was from Invitrogen Corp., and was used according to the manufacturer's instructions.)

(2) The ligation product was introduced into DH10B (Promega Co. Ltd) by electroporation (the electroporator was from Eppendorf Co. Ltd, and was operated at a voltage of 1800 V according to manufacturer's instruction), and the resulting bacteria were plated and cultured in LA resistant culture media containing 250 ppm kanamycin (Roche Co. Ltd) (for the formulation of LA, see Sambrook, J., E. F. Fritsch, and T. Maniatis, Molecular Cloning: a Laboratory Manual (3^{rd} edition), translated by Huang Peitang, Wang Jiaxi et al., Science Press (China), 2002 edition).

(3) The single colonies grown in the LA resistant culture media were inoculated in a laminar flow cabinet into 10 ml centrifuge tubes, which were prefilled with 3 ml of LB resistant culture media containing 250 ppm kanamycin, and then incubated on a shaker at 37° C. for 16-18 hours. Plasmids were extracted according to Sambrook J., and Russell D. W.—Molecular Cloning: A Laboratory Manual (translated by Huang Peitang et al., Science Press (China), 2002 edition), digested with KpnI and BamHI, and subjected to electrophoresis. Positive overexpression dual Ti plasmid vectors pU1301-HDT1 and pDS1301-HDT1-1 were obtained, based on the size of the insert.

(4) The T/A clone carrying the full-length OsHDT1 interfering fragment was digested with SpeI and SacI. The target fragment was recovered and ligated with plasmid pDS1301-HDT1-1 which has also been digested with SpeI and SacI. Following steps (2) and (3) described above, expression inhibitory vector pDS1301-HDT1-2 was obtained.

(5) The constructed expression vectors pU1301-HDT1 and pDS1301-HDT1-2 were introduced into Agrobacterium EHA105 strain (purchased from CAMBIA Corp.) by electroporation (reference and voltage used are as described above), and the transformed strains obtained were designated as TU-HDT1 and TR-HDT1.

### Example 3

### Transformation of Dual Ti Plasmid Vector and Detection of Gene Expressions and Copy Numbers in T0 Transgenic Plants

(1) TU-HDT1 and TR-HDT1 were transformed into rice recipient “Minghui 63” (obtained from Sanming Institute of Agricultural Science, Fujian, China) according to the method previously described by Hiei et al. (see Hiei et al., Efficient transformation of rice (Oryza sativa L.) mediated by Agrobacterium and sequence analysis of the boundaries of the T-DNA. Plant J, 1994, 6: 271-282). The resulting T0 transgenic plants were designated as PU-n and PR-n, respectively, wherein n is 1, 2, 3 . . . , representing different transgenic lines.

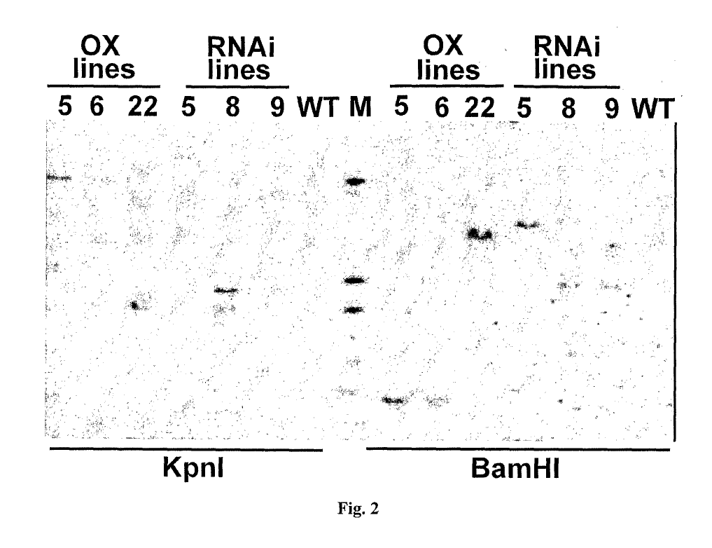

(2) The copy numbers in the transgenic plants were determined using Southern blotting (see Lu et al., Localization of pms3, a gene for photoperiod-sensitive genic male sterility, to a 28.4-kb DNA fragment. Mol Genet Genomics, 2005, 273: 507-511). Total DNA was digested overnight at 37° C. with suitable endonucleases. The digested fragments were separated on 1% (w/v) agarose gel and then transferred onto a nitrocellulose membrane. The probes were labeled by a random primer labeling protocol using isotopic label α-³²P-dCTP. The membrane was prehybridized in a hybridization tube for about 8-10 h. Then the isotopically labeled probes were added and hybridization was continued for 10 h. After hybridization was complete, the hybridized membrane was rinsed twice with 1×SSC, 0.1% SDS at ambient temperature, 5 minutes for the first time and 10 minutes for the second time. Then the membrane was hot-washed twice at 65° C. with a membrane washing solution of the same concentration, 10 minutes for each time. After washing, the membrane was placed on a clean filter paper and air dried. Then it was wrapped in a cling film, exposed to a phosphor screen and scanned to read the result. The used hybridization membrane was put into a radiation-proof dedicated device and allowed to naturally decay or washed with a probe washing solution to wash away the probes. The copy numbers were determined using hygromycin primers. The results showed that the PR-8 line had two copies, while other lines had only one copy, as shown in FIG. 2.

(3) The levels of expression of the target gene in the transgenic plants were determined using Northern blotting. The total RNA used was from leaves at tillering stage. The reagent used for RNA extraction was Trizol extraction kit from Invitrogen, and was used according to manufacturer's instruction. The loading amount for Northern membrane transfer was 15 μg, and the probes were labeled as described for Southern hybridization. The membrane was prehybridized in a hybridization tube for about 3 h. Then the isotopically labeled probes were added and hybridization was continued for 12 h. After hybridization was complete, the hybridized membrane was rinsed twice with 2×SSC, 0.1% SDS at ambient temperature, for 10 minutes each time. Then the membrane was hot-washed once at 65° C. with a membrane washing solution containing 0.5×SSC, 0.1% SDS for 3-5 minutes. If the hybridization signal was still very strong, the membrane was hot-washed once at 65° C. with a membrane washing solution containing 0.1×SSC, 0.1% SDS for 3 or more minutes until the hybridization signal was suitable. After washing, the membrane was placed on a clean filter paper and air dried. Then it was wrapped in a cling film, exposed to a phosphor screen and scanned to read the result. The used hybridization membrane was put into a radiation-proof dedicated device and allowed to naturally decay or hot-washed with 2×SSC at about 100° C. for 3 minutes to wash away the probes. The probes for OsHDT1 gene used in Northern blotting were the restriction fragments excised from plasmid PU1301-HDT1 using KpnI+PstI.

Seeds (T1 generation) were harvested from the T0 plants in preparation for field cultivation and hybridization.

In the present example, the media and reagents and the main steps used for genetic transformation (for obtaining transgenic plants) are as follows:

### (1) Abbreviations for Reagents and Solutions

The abbreviations for phytohormones used in culture media of the present disclosure are as follows: 6-BA (6-Benzylaminopurine); CN (Carbenicillin); KT (Kinetin); NAA (Naphthaleneacetic acid); IAA (Indoleacetic acid); 2,4-D (2,4-Dichlorophenoxyacetic acid); AS (Acetosyringone); CH (Casein Hydrolysate); HN (Hygromycin); DMSO (Dimethyl Sulfoxide); N6mac (macroelement solution for N6 basal medium); N6mic (microelement solution for N6 basal medium); MSmac (macroelement solution for MS basal medium); MSmic (microelement solution for MS basal medium)

### (2) Formulations of Primary Solutions

1) Preparation of macroelement mother solution for N6 basal medium (10× concentrate):

| | |
|---|---|
| Potassium nitrate (KNO₃) | 28.3 g |
| Potassium dihydrogen phosphate (KH₂PO₄) | 4.0 g |
| Ammonium sulfate ((NH₄)₂SO₄) | 4.63 g |
| Magnesium sulfate (MgSO₄•7H₂O) | 1.85 g |
| Potassium chloride (CaCl₂•2H₂O) | 1.66 g |

These compounds were dissolved in succession with distilled water and then the volume was brought to 1000 ml with distilled water at room temperature for later use.

2) Preparation of microelement mother solution for N6 basal medium (100× concentrate):

| | |
|---|---|
| Potassium iodide (KI) | 0.08 g |
| Boric acid (H₃BO₃) | 0.16 g |
| Manganese sulfate (MnSO₄•4H₂O) | 0.44 g |
| Zinc sulfate (ZnSO₄•7H₂O) | 0.15 g |

These compounds were dissolved in distilled water and then the volume was brought to 1000 ml with distilled water at room temperature for later use.

3) Preparation of iron salt (Fe₂EDTA) stock solution (100× concentrate):

800 ml double distilled water was prepared and heated to 70° C., then 3.73 g Na₂EDTA.2H₂O was added and fully dissolved. The resulting solution was kept in 70° C. water bath for 2 h, then brought to 1000 ml with distilled water and stored at 4° C. until use.

4) Preparation of vitamin stock solution (100× concentrate):

| | |
|---|---|
| Nicotinic acid | 0.1 g |
| Vitamin B1 (Thiamine HCl) | 0.1 g |
| Vitamin B6 (Pyridoxine HCl) | 0.1 g |
| Glycine | 0.2 g |
| Inositol | 10 g |

Distilled water was added to dissolve the compounds and the resulting solution was brought to 1000 ml with distilled water and stored at 4° C. until use.

5) Preparation of macroelement mother solution for MS basal medium (10× concentrate):

| | |
|---|---|
| Ammonium nitrate | 16.5 g |
| Potassium nitrate | 19.0 g |
| Potassium dihydrogen phosphate | 1.7 g |
| Magnesium sulfate | 3.7 g |
| Calcium chloride | 4.4 g |

These compounds were dissolved in distilled water and then the volume was brought to 1000 ml with distilled water at room temperature.

6) Preparation of microelement mother solution for MS basal medium (100× concentrate):

| | |
|---|---|
| Potassium iodide | 0.083 g |
| Boric acid | 0.62 g |
| Manganese sulfate | 0.86 g |
| Sodium molybdate (Na₂MoO₄•2H₂O) | 0.025 g |
| Copper sulphate (CuSO₄•5H₂O) | 0.0025 g |

These compounds were dissolved in distilled water and then the volume was brought to 1000 ml with distilled water at room temperature for late use.

7) Preparation of 2,4-D Stock Solution (1 mg/ml):

100 mg 2,4-D was weighed and dissolved in 1 ml 1 N potassium hydroxide for 5 minutes, then 10 ml distilled water was added for complete dissolution. The resulting solution was brought to 100 ml with distilled water and stored at room temperature until use.

8) Preparation of 6-BA Stock Solution (1 mg/ml):

100 mg 6-BA was weighed and dissolved in 1 ml 1 N potassium hydroxide for 5 minutes, then 10 ml distilled water was added for complete dissolution. The resulting solution was brought to 100 ml with distilled water and stored at room temperature until use.

9) Preparation of NAA Stock Solution (1 mg/ml):

100 mg NAA was weighed and dissolved in 1 ml 1 N potassium hydroxide for 5 minutes, then 10 ml distilled water was added for complete dissolution. The resulting solution was brought to 100 ml with distilled water and stored at 4° C. until use.

10) Preparation of IAA stock solution (1 mg/ml):

100 mg IAA was weighed and dissolved in 1 ml 1 N potassium hydroxide for 5 minutes, then 10 ml distilled water was added for complete dissolution. The resulting solution was brought to 100 ml with distilled water.

11) Preparation of glucose stock solution (0.5 g/ml):

125 g glucose was weighed and dissolved with distilled water. The resulting solution was brought to 250 ml with distilled water, sterilized and stored at 4° C. until use.

12) Preparation of AS stock solution:

0.392 g AS was weighed and dissolved in 10 ml DMSO. The resulting solution was dispensed into a 1.5 ml centrifuge tube and stored at 4° C. until use.

13) Preparation of 1 N potassium hydroxide stock solution:

5.6 g potassium hydroxide was weighed and dissolved in distilled water. The resulting solution was brought to 100 ml with distilled water and stored at room temperature until use.

### (3) Components and Amounts of Media for Genetic Transformation of Rice

### 1) Callus Induction Medium:

| | | |
|---|---|---|
| N6mac mother solution (10X) | 100 | ml |
| N6mic mother solution (100X) | 10 | ml |
| Fe²⁺ EDTA stock solution (100X) | 10 | ml |
| Vitamin stock solution (100X) | 10 | ml |
| 2,4-D stock solution | 2.5 | ml |
| Proline | 0.3 | g/L |
| CH | 0.6 | g/L |
| Sucrose | 30 | g/L |
| Phytagel | 3 | g/L |

Distilled water was added to a volume of 900 ml, and the pH value was adjusted to 5.9 with 1 N potassium hydroxide. The resulting mixture was boiled and brought to 1000 ml. The resulting medium was dispensed into 50 ml Erlenmeyer flasks (25 ml/flask), and the flasks were sealed and sterilized at 121° C. for 12 minutes.

### 2) Callus Subculture Medium:

| | | |
|---|---|---|
| N6mac mother solution (10X) | 100 | ml |
| N6mic mother solution (100X) | 10 | ml |
| Fe²⁺ EDTA stock solution (100X) | 10 | ml |
| Vitamin stock solution (100X) | 10 | ml |
| 2,4-D stock solution | 2.0 | ml |
| Proline | 0.5 | g/L |
| CH | 0.6 | g/L |
| Sucrose | 30 | g/L |
| Phytagel | 3 | g/L |

Distilled water was added to a volume of 900 ml, and the pH value was adjusted to 5.9 with 1 N potassium hydroxide. The resulting mixture was boiled and brought to 1000 ml. The resulting medium was dispensed into 50 ml Erlenmeyer flasks (25 ml/flask), and the flasks were sealed and sterilized at 121° C. for 12 minutes.

### 3) Pre-Culture Medium:

| | | |
|---|---|---|
| N6mac mother solution (10X) | 12.5 | ml |
| N6mic mother solution (100X) | 1.25 | ml |
| Fe²⁺ EDTA stock solution (100X) | 2.5 | ml |
| Vitamin stock solution (100X) | 2.5 | ml |
| 2,4-D stock solution | 0.75 | ml |
| CH | 0.15 | g/L |
| Sucrose | 5 | g/L |
| Agarose | 1.75 | g/L |

Distilled water was added to a volume of 250 ml, and the pH value was adjusted to 5.6 with 1 N potassium hydroxide. The resulting medium was sealed and sterilized at 121° C. for 12 minutes.

Prior to use, the medium was melted under heat and 5 ml glucose stock solution and 250 μl AS stock solution were added. The resulting medium was dispensed into Petri dishes (25 ml/dish).

### 4) Co-Culture Medium:

| | | |
|---|---|---|
| N6mac mother solution (10X) | 12.5 | ml |
| N6mic mother solution (100X) | 1.25 | ml |
| Fe²⁺ EDTA stock solution (100X) | 2.5 | ml |
| Vitamin stock solution (100X) | 2.5 | ml |
| 2,4-D stock solution | 0.75 | ml |
| CH | 0.2 | g/L |
| Sucrose | 5 | g/L |
| Agarose | 1.75 | g/L |

Distilled water was added to a volume of 250 ml, and the pH value was adjusted to 5.6 with 1 N potassium hydroxide. The resulting medium was sealed and sterilized at 121° C. for 12 minutes.

Prior to use, the medium was melted under heat and 5 ml glucose stock solution and 250 μl AS stock solution were added. The resulting medium was dispensed into Petri dishes (25 ml/dish).

### 5) Suspension Medium:

| | | |
|---|---|---|
| N6mac mother solution (10X) | 5 | ml |
| N6mic mother solution (100X) | 0.5 | ml |
| Fe²⁺ EDTA stock solution (100X) | 0.5 | ml |
| Vitamin stock solution (100X) | 1 | ml |
| 2,4-D stock solution | 0.2 | ml |
| CH | 0.08 | g/L |
| Sucrose | 2 | g/L |

Distilled water was added to a volume of 100 ml, and the pH value was adjusted to 5.4 with 1 N potassium hydroxide. The resulting medium was dispensed into two 100 ml Erlenmeyer flasks, and the flasks were sealed and sterilized at 121° C. for 12 minutes. Prior to use, 1 ml glucose stock solution and 100 μl AS stock solution were added.

### 6) Selection Medium:

| | | |
|---|---|---|
| N6mac mother solution (10X) | 25 | ml |
| N6mic mother solution (100X) | 2.5 | ml |
| Fe²⁺ EDTA stock solution (100X) | 2.5 | ml |
| Vitamin stock solution (100X) | 2.5 | ml |
| 2,4-D stock solution | 0.625 | ml |
| CH | 0.15 | g/L |
| Sucrose | 7.5 | g/L |
| Agarose | 1.75 | g/L |

Distilled water was added to a volume of 250 ml, and the pH value was adjusted to 6.0 with 1 N potassium hydroxide. The resulting medium was sealed and sterilized at 121° C. for 12 minutes. Prior to use, the medium was melted and 250 μl HN and 400 ppm CN were added. The resulting medium was dispensed into Petri dishes (25 ml/dish).

### 7) Pre-Differentiation Medium:

| | | |
|---|---|---|
| N6mac mother solution (10X) | 25 | ml |
| N6mic mother solution (100X) | 2.5 | ml |
| Fe²⁺ EDTA stock solution (100X) | 2.5 | ml |
| Vitamin stock solution (100X) | 2.5 | ml |
| 6-BA stock solution | 0.5 | ml |
| KT stock solution | 0.5 | ml |
| NAA stock solution | 50 | μl |
| IAA stock solution | 50 | μl |
| CH | 0.15 | g/L |
| Sucrose | 7.5 | g/L |
| Agarose | 1.75 | g/L |

Distilled water was added to a volume of 250 ml, and the pH value was adjusted to 5.9 with 1N potassium hydroxide. The resulting medium was sealed and sterilized at 121° C. for 12 minutes. Prior to use, the medium was melted and 250 μl HN and 200 ppm CN were added. The resulting medium was dispensed into Petri dishes (25 ml/dish).

### 8) Differentiation Medium:

| | | |
|---|---|---|
| N6mac mother solution (10X) | 100 | ml |
| N6mic mother solution (100X) | 10 | ml |
| Fe²⁺ EDTA stock solution (100X) | 10 | ml |
| Vitamin stock solution (100X) | 10 | ml |
| 6-BA stock solution | 2 | ml |
| KT stock solution | 2 | ml |
| NAA stock solution | 0.2 | ml |
| IAA stock solution | 0.2 | ml |
| CH | 1 | g/L |
| Sucrose | 30 | g/L |
| Phytagel | 3 | g/L |

Distilled water was added to a volume of 900 ml, and the pH value was adjusted to 6.0 with 1N potassium hydroxide. The resulting mixture was boiled, brought to 1000 ml and dispensed into 50 ml Erlenmeyer flasks (50 ml/flask). The flasks were sealed and sterilized at 121° C. for 12 minutes.

### 9) Rooting Medium:

| | | |
|---|---|---|
| MSmac mother solution (10X) | 50 | ml |
| MSmic mother solution (100X) | 5 | ml |
| Fe²⁺ EDTA stock solution (100X) | 5 | ml |
| Vitamin stock solution (100X) | 5 | ml |
| Sucrose | 30 | g/L |
| Phytagel | 3 | g/L |

Distilled water was added to a volume of 900 ml, and the pH value was adjusted to 5.8 with 1N potassium hydroxide. The resulting mixture was boiled, brought to 1000 ml and dispensed into rooting tubes (25 ml/tube). The tubes were sealed and sterilized at 121° C. for 12 minutes.

### (4) Procedure of Genetic Transformation Mediated by Agrobacterium

### 4.1 Callus Induction

1) Mature rice seeds of “Minghui 63” (obtained from Sanming Institute of Agricultural Science, Fujian, China) were husked, treated with 70% alcohol for 1 minute and surface-disinfected with 0.15% HgCl₂ for 15 minutes;

2) The seeds were rinsed with sterilized water for 4-5 times;

3) The sterilized seeds were put onto the induction medium (the formulation of the induction medium was as described above);

4) The seeded medium was placed in darkness for 4-week culture at 25±1° C. to obtain rice calli.

### 4.2 Callus Subculture

Bright yellow, compact and relatively dry embryogenic calli were selected, seeded onto subculture medium as described above, and cultured in darkness for 2 weeks at 26±1° C. to obtain subcultured rice calli.

### 4.3 Callus Pre-Culture

The compact and relatively dry embryogenic calli were selected, seeded onto the pre-culture medium as described above, and cultured in darkness for 4 days at 26±1° C.

### 4.4 Agrobacterium Culture

1) Agrobacterium EHA105 was inoculated and pre-cultured on the LA culture medium with corresponding resistance selection at 28° C. for 48 hours;

2) The Agrobacterium from the step 1) was transferred to the suspension medium as described above and cultured on a shaker at 28° C. for 2-3 hours.

### 4.5 Agrobacterium Infestation

1) The pre-cultured calli were transferred into sterilized glass flasks;

2) The Agrobacterium suspension was adjusted to OD₆₀₀ 0.8-1.0;

3) The calli were immersed in the Agrobacterium suspension for 30 minutes;

4) The calli from step 3) were transferred onto a sterilized filter paper and dried, and then seeded onto the co-culture medium as described above and cultured for 72 hours (3 days) culture at 19-20° C.

### 4.6 Washing and Selective Culture of Calli (Screening for Resistance)

1) The rice calli were washed with sterilized water until no Agrobacteria were observed;

2) The calli from step 1) were immersed in sterilized water containing 400 ppm carbenicillin (CN) for 30 minutes;

3) The calli from step 2) were transferred onto a sterilized filter paper so that the calli were free of water;

4) The calli from step 3) were transferred onto the selection medium and screened for 2-3 times, 2 weeks for each time. (The concentration of hygromycin was 400 mg/L for the first screen and 250 mg/L for later screens).

### 4.7 Pre-Differentiation and Differentiation

1) The resistant calli obtained were transferred to the pre-differentiation medium as described above, and cultivated in darkness for 5-7 days at 26° C.;

2) The pre-differentiated calli were transferred to the differentiation medium as described above, and cultivated under light at a light intensity of 2000 1× at 26° C. for about 5 weeks to obtain transgenic plantlets with a few roots.

### 4.8 Induction of Rooting

1) The roots of the above transgenic plantlets were cut off;

2) The plantlets were then transferred to the rooting medium as described above, and cultivated at a light intensity of 2000 1× at 26° C. for about 18 days to obtain transgenic rice plantlets that grow roots.

### 4.9 Transplantation

The residual medium on the roots of the plantlets was washed off, and these plantlets with good root system were transferred into a greenhouse. The greenhouse was maintained humidified in the first few days of transplantation.

### Example 4

### Detection for T1 Positive Transgenic Plants and Cross in Field

T1 seeds were collected from the following OHDT1 transgenic plants and planted in field: overexpression PU negative lines (three: PU29, PU30 and PU31) and positive lines (three: PU5, PU6 and PU22), as well as RNAi-inhibited expression PR negative lines (three: PR6, PR14 and PKG, which were introduced with empty RNAi vector) and positive lines (three: PR1, PR8 and PR9).

(1) The T1 plants from the positive lines were detected for positive plants at DNA level as follows. Total DNA was extracted from the leaves using CTAB method (Murray et al., Rapid isolation of high molecular weight plant DNA. Nucleic Acids Res, 1980, 8: 4321-4325). T1 transformant plants were detected for positive plants using hygromycin primers by a common PCR method. The hygromycin primers were Hn-F and Hn-R (provided by Shanghai Shenggong Biotechnology Co., Ltd, Shanghai, China) with the sequences shown in Table 4. PCR reaction was conducted in a total volume of 20 μl, which consisted of 100 ng of templates, 2 μl of 10×PCR buffer, 1.6 μl of 10 mM dNTPs, 1.5 μl of 2.5 mM Mg²⁺, 0.4 μl each of forward and reverse primers, 0.2 μl of Taq enzyme and water which was added to make up the volume of 20 μl (the PCR buffer, dNTPs, Mg²⁺, rTaq enzyme used were all purchased from TAKARA Co., Ltd). The conditions of PCR reaction were as follows: (1) 94° C. for 4 min; (2) 32 cycles of 94° C. for 1 min, 56° C. for 1 min, and 72° C. for 2.5 min; and (3) 72° C. for 10 min; and (4) 4° C. storage. The PCR products were detected via electrophoresis on 1% agarose gel. The transgenic plants with the band characteristic of the hygromycin gene were positive plants, while the plants without said band were negative plants, as the hygromycin gene was specific for the transformation vector.

(2) The positive plants from each of the lines described above, as well as wild-type “Minghui 63”, were selected as male parents to cross with female parent “Zhenshan 97A” (obtained from Jiangxi Academy of Agricultural Sciences, Jiangxi, China). Seeds of both the hybrid progenies and the self-bred progenies were harvested.

### Example 5

### Field Observation on Traits of Hybrid Progenies and Self-Bred Progenies

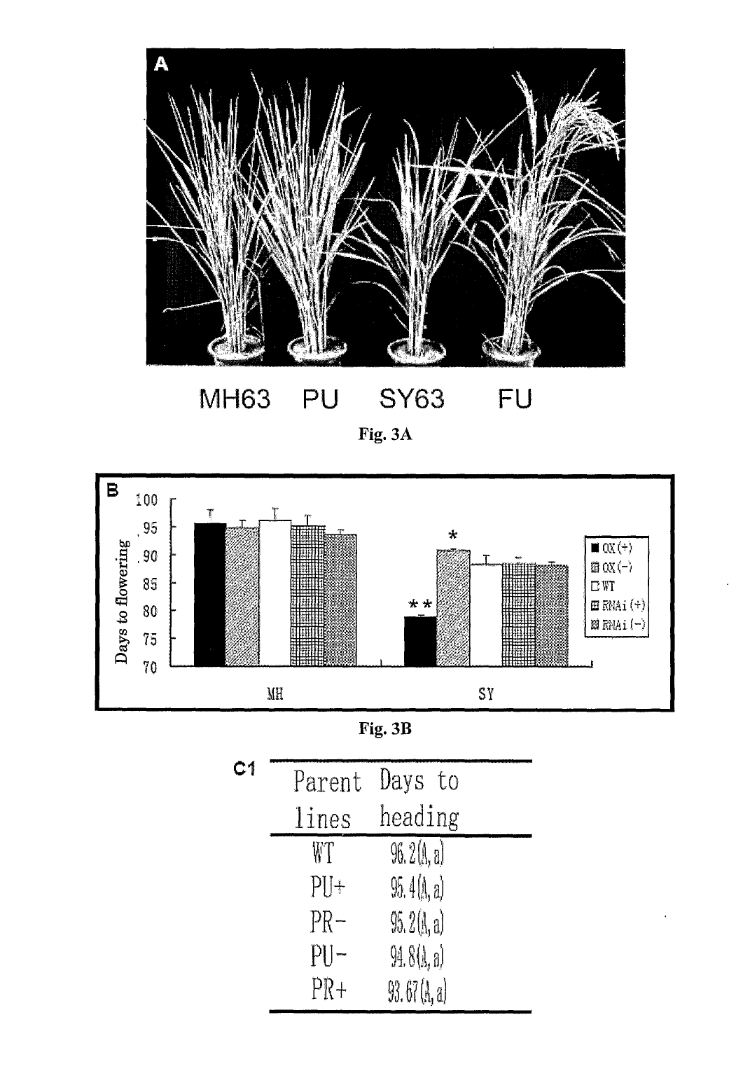

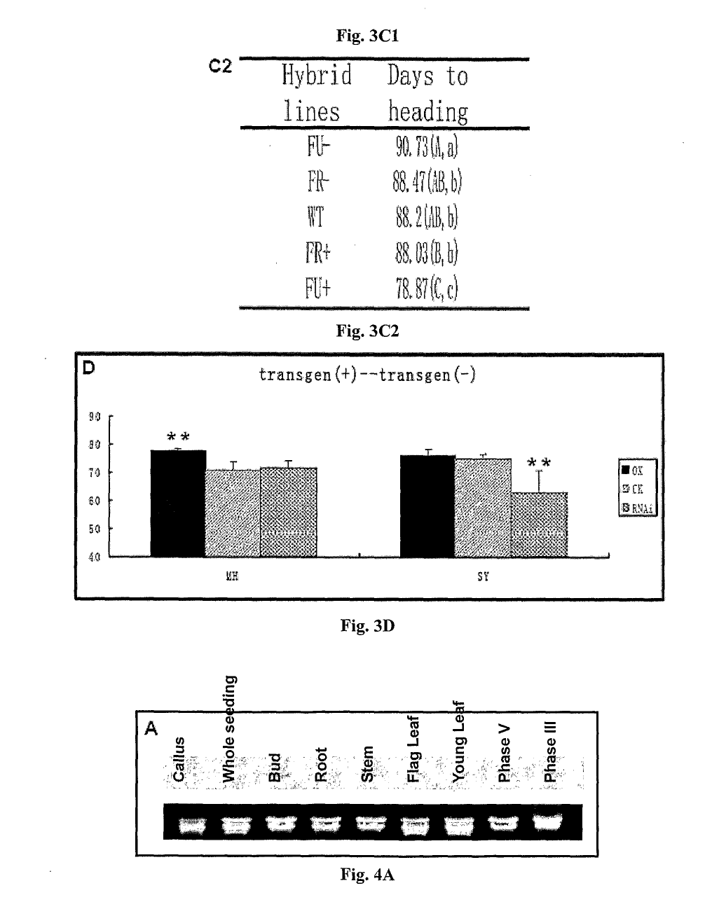

The seeds of both the hybrid progenies and the self-bred progenies harvested as described in Example 4 above were planted in field in two portions on May, 2006 and May, 2008. Each line was planted in triplicate with 10×6 plants for each of triplicate. The 8×4 plants in the middle were selected for phenotype observation and trait investigation. The data obtained from trait investigation were subjected to variance analysis using SAS software. The results indicated that the hybrid overexpression plants exhibited an obvious early-flowering trait compared to the wild-type hybrid plants and the negative control plants, while the transgenic and wild-type parent plants showed no distinct difference in flowing period. Moreover, the RNAi inhibition hybrid plants had a decreased seed setting rate compared to the hybrid negative control plants, while the RNAi inhibition parent plants and negative control plants showed no evident difference in the trait of seed seeting rate (as shown in FIG. 3).

### Example 6

### Expression Pattern and Subcellular Localization of Rice Endogenous OsHDT1 Gene

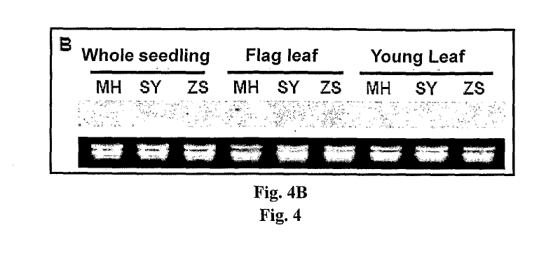

(1) Samples were taken from calli, roots, buds, young leaves, flag leaves, whole seedlings, and phase III and, phase V young-ears of the plants of rice variety “Minghui 63”. Total RNA was extracted as described in Example 3 above and Northern blotting was performed to determine the levels of expression of the gene in various parts of the plants. Results showed that OsHDT1 gene had a higher level of expression in buds, sterns, and phase III and phase V young-ears, and a lower level of expression in young leaves, flag leaves and whole seedlings (see FIG. 4A).

Moreover, in order to determine whether the gene cloned according to the present disclosure exhibited different expression in different rice varieties, the present inventors selected rice varieties “Minghui 63” and “Zhenshan 97” and their hybrid progeny “Shanyou 63”, and performed Northern blotting on total RNA extracted from young leaves, flag leaves and whole seedlings. The results, as shown in FIG. 4B, showed that there was essentially no difference in the levels of expression in the three parts of the plants.

(2) Subcellular localization of the gene was investigated by particle bombardment using vector pU1391GFP, as described in Huang et al., Down-regulation of a Silent Information Regulator2-related gene, OsSRT1, induces DNA fragmentation and cell death in rice. Plant Physiol, 2007, 144:1508-1519. In particular, the full-length encoding sequence (with stop codon excluded) of the gene was amplified by PCR and ligated into vector pU1391GFP in-frame with GFP (green fluorescent protein). Particle bombardment was performed as follows. The contructed plasmid DNA (5 μg) was mixed with 3 mg of gold powder having a particle size of 1 μm. The mixture was suspended in 60 μl of absolute alcohol, and divided into five aliquots for performing particle bombardment. Prior to particle bombardment, onion epidermis was peeled off and cut into pieces of about 1 cm² which were then tightly laid onto a moistened Petri dish. Particle bombardment was performed using PDS-1000 System (BioRad Co. Ltd) at a helium pressure of 1100 psi. The bombarded pieces of onion epidermis were cultured at 25° C. in dark for 24 hours followed by observation for the site of expression of GFP in the onion epidermal cells using a confocal microscope from Leica Co., Ltd.

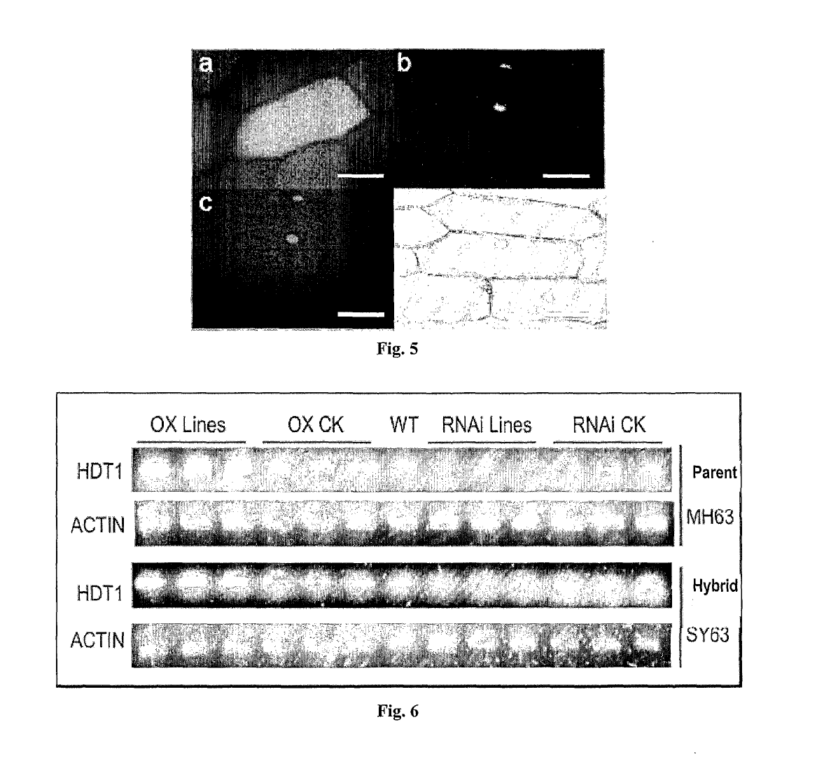

The primers used for subcellular localization in this Example were HDTNLS-F and HDTNLS-R shown in FIG. 2. Results showed that HDT1::GFP was localized in the nucleus of the onion epidermal cells (see FIG. 5).

### Example 7

### Analysis of Expression of OsHDT1 Gene and Investigation of Protein Function

The expression of OsHDT1 gene in the transgenic and wild-type parent and hybrid plants were analysed using RT-PCR to determine whether there were differences in expression in these plants. The total RNA used in the study were from leaves at rice tillering stage. The reagent used for RNA extraction was Trizol extraction kit from Invitrogen, and was used according to manufacturer's instruction. The reverse transcriptional synthesis of the first strand of cDNA in RT-PCR was conducted as follows. (1) Preparing Mixture 1: to 2 μg of total RNA, 2U of DNAse I and 1 μl of 10× DNAse I buffer, DEPC (diethyl pyrocarbonate, a strong inhibitor of RNAse)-treated water (0.01% DEPC) was added to a volume of 10 μl and mixed. The resulting Mixture 1 was placed at 37° C. for 20 minutes to remove DNA. (2) 20 minutes later, Mixture 1 was incubated in a 70° C. water bath for 10 minutes to remove DNAse I activity, followed by placing on ice for 5 minutes. (3) 1 μl of 500 μg/ml oligo(dT) was added into Mixture 1. (4) The cooled Mixture 1 was immediately placed in a 70° C. water bath for 10 minutes to completely denature RNAs, followed by placing on ice for 5 minutes. (5) Preparing Mixture 2: 10 μl of Mixture 1, 4 μl of 5× first strand buffer, 2 μl of 0.1 M DTT (dithiothreitol), 1.5 μl of 10 mM dNTP mixture, 0.5 μl of DEPC-treated water and 2 μl of reverse transcripatase were mixed to obtain Mixture 2, which was then incubated in a 42° C. water bath for 1.5 hours. (6) After reaction was complete, Mixture 2 was placed on a 90° C. dry bath for 3 minutes. (7) The final reaction product was stored at −20° C. The reagents used in the reaction were all purchased from Invitrogen Corp. RT-PCR reaction was conducted in a volume of 20 μl, which consisted of 1 μl of the template for the first strand of cDNA, 2 μl of 10×PCR buffer, 1.6 μl of 10 mM dNTPs, 1.5 μl of 2.5 mM Mg²⁺, 0.4 μl each of left and right primers, 0.2 μl of Taq enzyme and water which was added to make up the volume of 20 μl (the PCR buffer, dNTPs, Mg²⁺ and rTaq enzyme used were all purchased from TAKARA Co., Ltd, or TAKARA Biotechnology (Dalian) Co., Ltd). The conditions of PCR reaction were as follows: (1) 94° C. for 2 min; (2) 30 cycles of 94° C. for 1 min, 58° C. for 1 min, and 72° C. for 2 min; and (3) 72° C. for 7 min; and (4) 4° C. storage. The PCR products were detected by electrophoresis on 1.2% agarose gel. The primers of OsELP3 gene used in RT-PCR were RTHDT-F and RTHDT-R, and the primers of Actin gene were Actin-F and Actin-R, their sequences being shown in Table 4.

The results, as shown in FIG. 6, indicated that the levels of expression in the transgenic and wild-type parent and hybrid plants were as expected. The overexpression plants showed overexpression effect and the RNAi plants showed inhibitory effect. There were no obvious differences in expression between the corresponding parent and hybrid plants.

In order to determine the effect of HDT1 protein on histone modification and the difference in levels of modification between the parent and hybrid plants, histone was extracted from transgenic and wild-type parent and hybrid OsHDT1 plants to perform Western blotting and investigate the function of HDT1 protein using commercial antibodies against histone modifications, as described in Huang et al., Down-regulation of a Silent Information Regulator2-related gene, OsSRT1, induces DNA fragmentation and cell death in rice. Plant Physiol, 2007, 144:1508-1519. In particular, 4-5 pieces of leaves were taken from each of the wild-type and transgenic rice plants about 40 days after sowing. The leaves was triturated in liquid nitrogen and mixed thoroughly in a 3-fold volume of histone extraction buffer (10 mM Tris-HCl, pH 7.5; 2 mM EDTA; 0.25 M HCl; 5 mM 2-mercaptoethanol; 0.2 mM PMSF). The sample was centrifuged at 12,000 g at 4° C. for 10 minutes. The supernatant were pipetted into a new centrifuge tube and trichloroacetic acid was added to a final concentration of 25%. The sample was centrifuged at 17,000 g at 4° C. for 30 minutes and the supernatant was discarded. The pellets were wash three times with acetone, air-dried and dissolved in loading buffer (62.5 mM Tris-HCl, pH 6.8; 2% SDS; 25% glycerol; 0.01% Bromophenol Blue; 10% β-mercaptoethanol). The sample was subjected to SDS-PAGE electrophoresis (15%) to determine the extraction efficiency using Mini-PROTEAN3 electrophoresis cell system from Bio-Rad Co., Ltd according to the manufacturer's instruction.

The extracted histone was subjected to Western blotting. In particular, the extracted histone was transferred to Hybond-P PVDF membrane from Amersham Co., Ltd using Mini Trans-Blot Cell from Bio-Rad Co., Ltd according to the manufacturer's instruction. The membrane was blocked for more than two hours with a previously prepared PBS buffer (NaCl 137 mmol/L, KCl 2.7 mmol/L, Na₂HPO₄ 4.3 mmol/L, KH₂PO₄ 1.4 mmol/L, pH 7.5) containing 2% w/v bovine serum albumin (BSA). The membrane was incubated at room temperature with different antibodies added at a 1:10000 dilution for about two hours. The antibodies used were anti-H4K16 (07-329), anti-H4ace (06-866), anti-H3ace (06-599) antibodies from Upstate Corp., and anti-H3 (ab1791) and anti-H4K5 (ab51997) antibodies from Abcam Co., Ltd (the numbers in parentheses are product numbers). After pouring off the solution of primary antibodies, the membrane was washed with PBS buffer for three times, 15 minutes for each time. Then HRP-labeled goat anti-rabbit secondary antibody (SouthernBiotech Corp.) at a 1:10000 dilution (v/v) was added followed by incubation at room temperature for 1-2 hours. The membrane was then washed with PBS buffer for three times, 15 minutes for each time. Finally, X-ray film was developed using SuperSingnal Pico kit (Pierce Co., Ltd) according to manufacturer's instruction and scanned with a scanner for analysis.

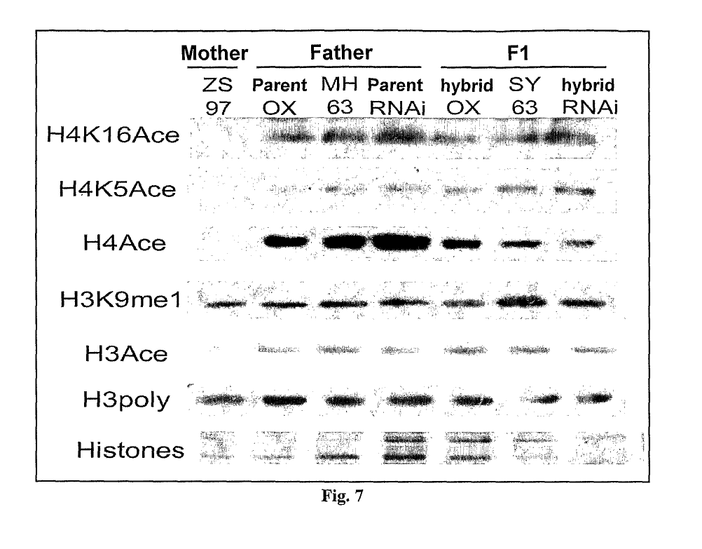

The results obtained, as shown in FIG. 7, indicated that HDT1 was capable of deacetylating histone and as such is a histone deacetylase mainly acting at H4K16 site. In addition, there existed significant differences in the level of histone modification among “Minghui 63”, “Zhenshan 97” and “Shanyou 63”, with “Zhenshan 97” having a very low level of histone modification, which might be one of the reasons for its male sterility.

TABLE 2
The sequences of the primer pairs used in Examples

| Gene | Name of Primers | Sequences of Primers |
|---|---|---|
| OsHDT1 | FLHDT-F | GGGGGTACCCCGATTCCGATGGAGTTCTG (SEQ ID |
| | | NO: 3) |
| | FLHDT-R | GGGGGATCCGAAGCTCAGTTCGCACCACAG (SEQ ID |
| | | NO: 4) |
| | HDTRNAi-F | GGGACTAGTGGTACCGGCTGCAGTGAATGACGATG |
| | | (SEQ ID NO: 5) |
| | HDTRNAi-R | GGGGAGCTCGGATCCTCACTTGGCGGGGTGCTTGG |
| | | (SEQ ID NO: 6) |
| | HDTNLS-F | GGGGAATTCCCGATTCCGATGGAGTTCTG (SEQ ID |
| | | NO: 7) |
| | HDTNLS-R | GGGGGATCCCTTGGCGGGGTGCTTGGCCT (SEQ ID |
| | | NO: 8) |
| | RTHDT-F | CGCTTTTTGCACCTTTCTCAG (SEQ ID NO: 9) |
| | RTHDT-R | ACTTTGCCATTTGCCCTGG (SEQ ID NO: 10) |
| Hygromycin | Hn-F | AGAAGAAGATGTTGGCGACCT (SEQ ID NO: 11) |
| | Hn-R | GTCCTGCGGGTAAATAGCTG (SEQ ID NO: 12) |
| actin | Actin-F | TATGGTCAAGGCTGGGTTCG (SEQ ID NO: 13) |
| | Actin-R | CCATGCTCGATGGGGTACTT (SEQ ID NO: 14) |

Note:
the above primers were all synthesized by Shanghai Shenggong Biotechnology Co., Ltd, Shanghai, China.

### Example 8

### Expression Analysis of Genes Associated with Flowering Time in Transgenic Plants

As the overexpressing plants had varied flowering time, the present inventors investigated the circadian expression rhythm of the gene according to the present disclosure and other genes associated with flowering time, as well as the difference in levels of expression of these genes in parent and hybrid overexpressing and wild-type plants. This was done as follows. The seeds from “Minghui 63” and “Shanyou 63” wild-type and overexpressing plants as well as “Zhenshan 97” wild-type plants were germinated. One week later, the young seedlings, which were divided into two groups, were transferred to a bread bin containing soil which was then placed into an artificial climate chamber (Versatile Environmental Test Chamber MLR-351H, SANYO) for cultivation with the following settings: light for 15 h, temperature 30° C.; absence of light for 9 h, temperature 25° C.; constant humidity of 70%. After cultivation under long sunlight condition for 3 weeks, one groups of growing plants were transferred to short sunlight condition and cultivated with the following settings: light for 9 h, temperature 30° C.; absence of light for 15 h, temperature 25° C.; constant humidity of 70%. The other groups of growing plants continued to be cultivated under long sunlight condition. After a further cultivation for 2 weeks, samples were taken from each groups of the plants at 4 hours interval for 24 hours. Total RNAs were extracted from the samples and reverse transcribed. The transcription products were subjected to quantitative PCR in a 25 μL reaction system containing 1.5 μL of the transcription products, 0.25 μM of each of left and right primers and 12.5 μL of SYBR Green Mix (Takara). The reaction was performed on a 7500 real-time quantitative PCR instrument (Takara) according to the manufacturer's instruction of Takara Co. Ltd, with rice actin1 gene (Xue et al., Natural variation in Ghd7 is an important regulator of heading date and yield potential in rice. Nat Genet, 2008, 40: 761-7) as an internal reference for the reaction. The reaction comprised 40 cycles. Each sample was run in triplicate and normalized to the expression level of actin1.

The present inventors first studied the expression pattern of OsHDT1 gene in three wild-type plants of each of “Minghui 63” and “Shanyou 63”. Results showed that the OsHDT1 gene cloned according to the present disclosure had essentially the same expression pattern in these two rice varieties under long and short sunlight conditions, exhibiting a clear circadian rhythm. In particular, under long sunlight condition, the expression level of OsHDT1 rose continuously during the daytime, reaching a peak at 16:30 (4 hours before dark), followed by continuous decline; while under short sunlight condition, the expression level increased incessantly during the daytime, reaching peak at 20:30 (4 hours after dark), and then fell continuously.

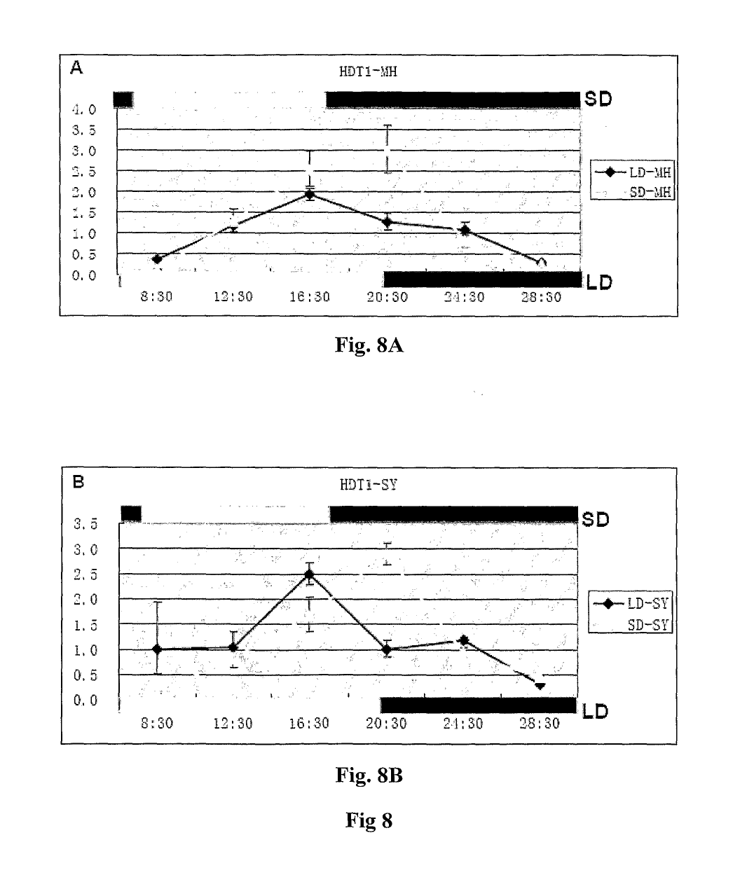

The expression level rose with light induction, reached a peak three hours after disappearance of light, and then declined, reaching the lowest level at half of an hour after generation of light (the results are as shown in FIGS. 8A and B). The primes for OsHDT1 used in the quantitative PCR are shown in Table 2.

Flowering genes Hd3a, Hd1, Ehd1 and OsGI of rice (Xue et al., Natural variation in Ghd7 is an important regulator of heading date and yield potential in rice. Nat Genet, 2008, 40: 761-7) were assayed for their levels of expression in “Minghui 63” and “Shanyou 63” OsHDT1-overexpressing transgenic plants and wild-type plants under long and short sunlight conditions using reverse transcription (RT) products from the plants treated above. The primers used in PCR for each of the genes are shown in Table 2.

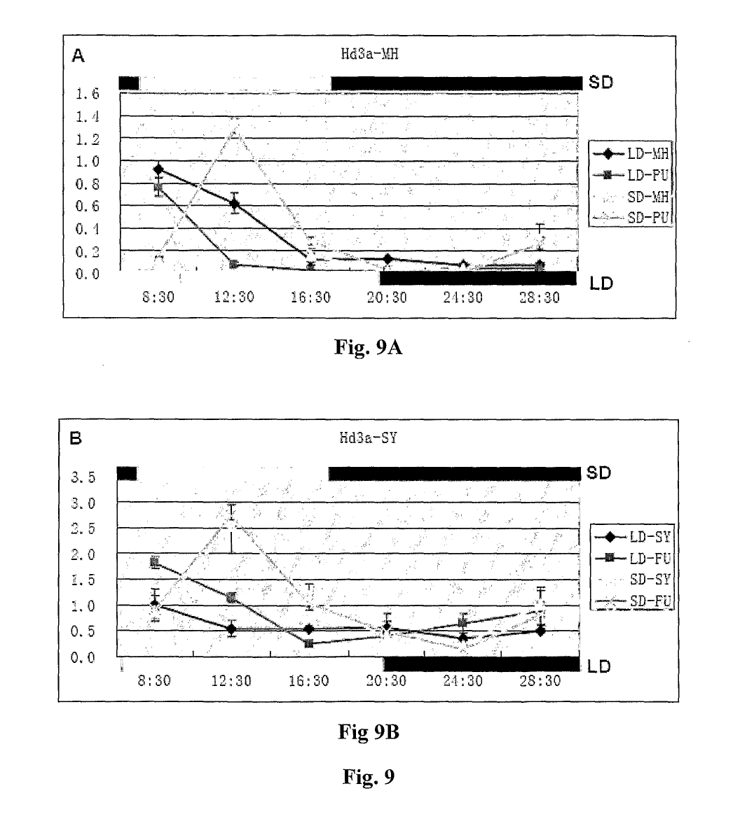

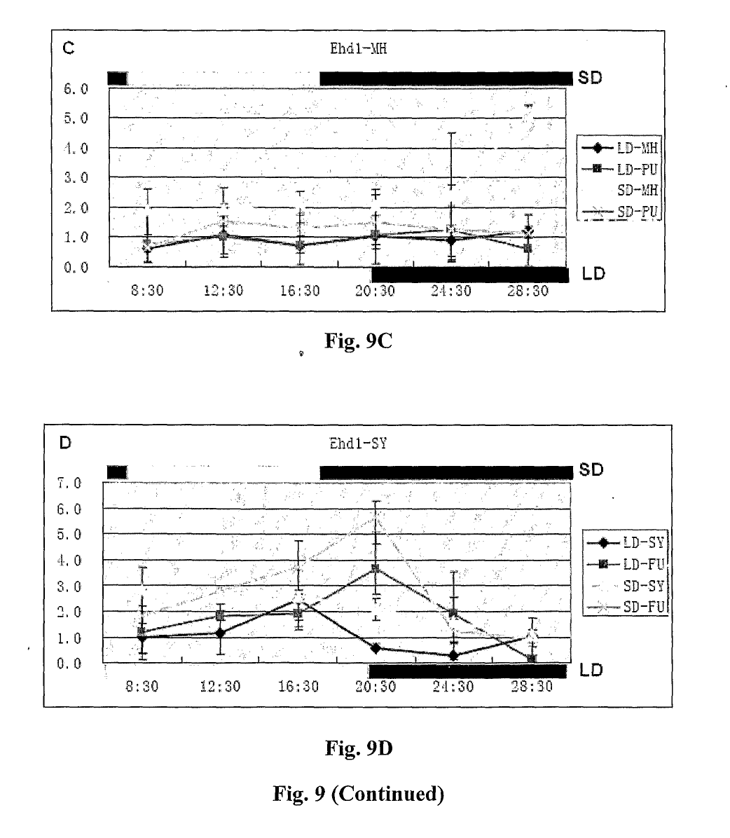

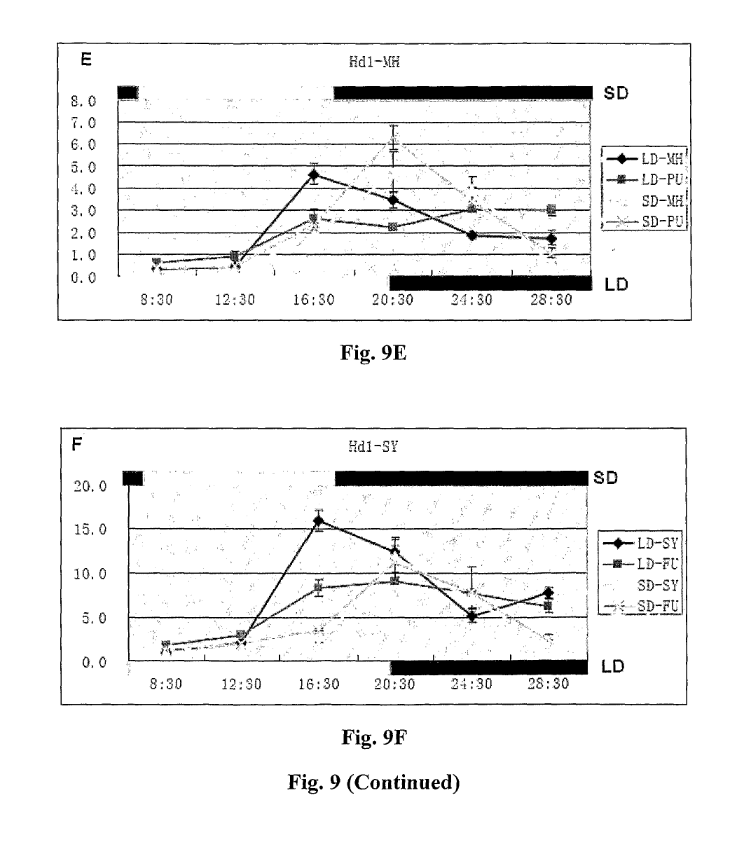

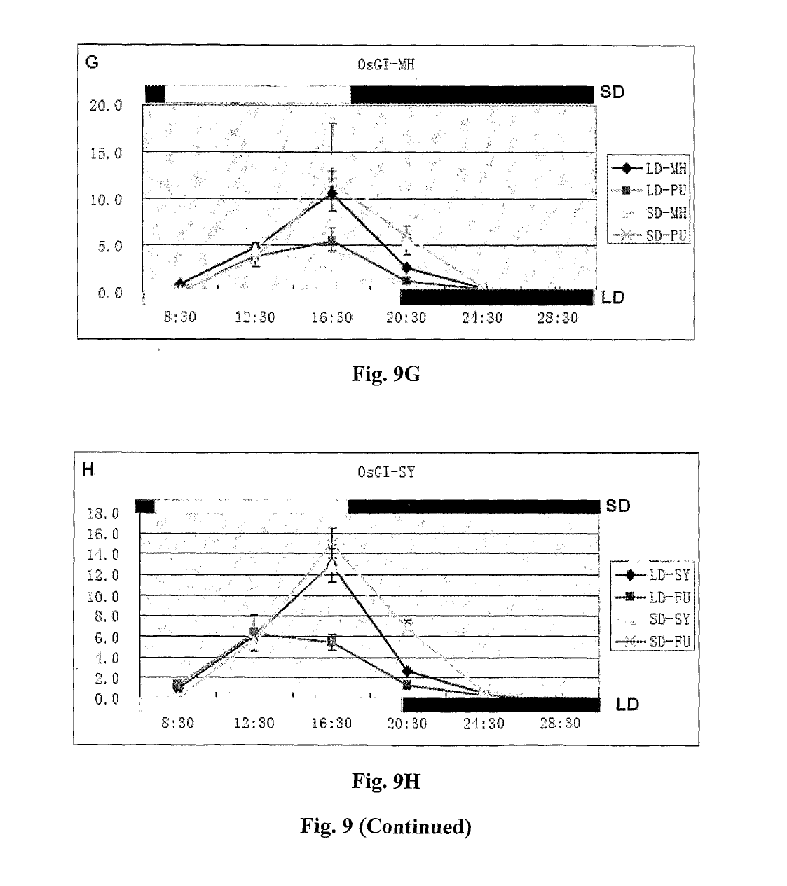

Results showed that, under long sunlight condition, the peak expression (at 8:30, 4 hours after lighting) of Hd3a in FU was higher than in SY (see FIG. 9B), while for MH63 plants, there was no significant difference in the expression between overexpressing plants and wild-type plants (see FIG. 9A). This was consistent with the phenotypes.

In FU, Ehd1 had a suddenly increased level of expression upon stop of lighting, which subsequently returned to normal (see FIG. 9D), while in MH63 overexpressing plants, the gene showed no significant difference in expression in comparison to wild-type plants (see FIG. 9C).

The peak expression (at 16:30, 4 hours before dark) of Hd1 in FU was lower than in SY (see FIG. 9F), while in MH63 plants, although the peak expression of the overexpressing plants was lower than that of wild-type plants, the difference was not as significant (see FIG. 9E).

OsGI had a similar expression pattern to Hd1, with a lower expression in FU than in SY (see FIG. 9H) and a slightly lower expression in PU than in MH (see FIG. 9G) at 16:30.

It was therefore believed that OsHDT1 influences the flowering time of rice by inhibiting the expression of genes Hd1 and OsGI and thereby increasing the expression of gene Hd3a. However, as the light cycle regulation pathway begins from Ehd1, the expression patterns of the genes in “Minghui 63” and “Shanyou 63” deviated from each other. In particular, OsGI and Hd1 had changed expression in both parent and hybrid overexpressing plants, while Ehd1 and Hd3a only had changed expression in “Shanyou 63” overexpressing plants, but not in “Minghui 63” overexpressing plants, which ultimately resulting in the phenomenon that the “Minghui 63” plants had early flowering time but the “Shanyou 63” plants did not have this phenotype.

### Example 9

### Analysis of Expression Profiles of Histone Acetylase and Deacetylase in Rice

The gene sequences of histone acetylase and deacetylase in rice were all obtained by searching the public database ChromDB (http://www.chromdb.org/). The search was done by entering the “Advanced Search” interface from “Search Tools” on the homepage, and then selecting the intended organism and protein group. As the present disclosure relates to indica rice, “Oryza sativa (indica cultivar-group)” was selected as the organism.

According to relevant literature, histone acetylase genes can be categorized into the following four families: HAC family [CREBBP(CBP) Family], HAF family [TATA Box Binding Protein Associated Factors (TAFI Homologs)], HAG family (GNAT Superfamily) and HAM family (MYST Family). Additionally, there exists an interaction protein HXA family [Complex component (Ada2 homologs)] that binds with acetylases. The names of these five families were used as options for “Protein Group” in the above said database to conduct searches to obtain histone acetylase genes in rice.

Histone deacetylase genes can be categorized into the following three families: HAD family [Rpd3/HDA1 superfamily], HDT family (Plant-specific HD2 Family) and SRT family (SIR2 Family). Additionally, there exists an interaction protein SNT family [Complex protein (Sin3 homologs)] that binds with deacetylases. The names of these four families were used as options for “Protein Group” in the above said database to conduct searches to obtain histone deacetylase genes in rice.

The information obtained from the searched genes is shown in the following table.

TABLE 3
Histone acetylase and deacetylase genes found in this Example

| | | | | | |
|---|---|---|---|---|---|
| Gene | Gene | Chrome DB ID | Chrome DB | | |
| family | name | (Japonica) | ID (Indica) | Gene ID | Chromosome |

Histone Acetylase
CREBBP(CBP)

| | | | | |
|---|---|---|---|---|
| HAC | HAC701 | HAC2201 | 3032923 4326755 | Os01g14370 |
| | HAC703 | HAC2202 | 3057806 4328238 | Os02g04490 |
| | HAC704 | HAC2203 | 4341999 | Os06g49130 |

TATA Box Binding Protein Associoted Factors (TAFI Homologs)

| | | | | |
|---|---|---|---|---|
| HAF | HAF701(HAC713) | HAF2201 | 4341656 | Os06g43790 |

GNAT superfamily

| | | | | | |
|---|---|---|---|---|---|
| HAG | GCN5 | HAG702(HAC705) | HAG2201 | 3053264 4348629 | Os10g28040 |
| | ELP3 | HAG703(HAC706) | HAG2202 | 3035426 4336205 | Os04g40840 |
| | HATb | HAG704 | HAG2203 | 4346812 | Os09g17850 |
| MYST | | | | | |
| HAM | MYST | HAM701(HAC702) | HAM2201 | 3043281 4343971 | Os07g43360 |

Complex component (Ada2 homologs)

| | | | | |
|---|---|---|---|---|
| HXA | ADA2 | HXA701 | 3064873 4334126 | Os03g53960 |

Histone Deacetylase
Plant-specific HD2 Family

| | | | | | |
|---|---|---|---|---|---|
| HDT | HDT1(HD2b) | HDT701 | HDT2201 | 3068614 4339823 | Os05g51830 |
| | HDT2(HD2a) | HDT702 | HDT2202 | | Os01g68104 |

Rpd3/HDA1 superfamily

| | | | | | |
|---|---|---|---|---|---|
| Class I | | HDA701 | HDA2201 | 3047027 | Os01g40400 |
| | HDAC1 | HDA702 | HDA2205 | 4341387 | Os06g38470 |
| | | HDA703 | HDA2207 | 3058668 3058671 | Os02g12350 |
| | | | | 4328717 | |
| | | HDA705 | HDA2206 | 3072424 4345332 | Os08g25570 |
| | HDA19 | HDA707 | | | Os01g12310 |
| | | HDA709 | HDA2202 | 4350006 | Os11g09370 |
| | | HDA710 | HDA2204 | 3058675 3058676 | Os02g12380 |
| | | | | 3058677 4328720 | |
| | | HDA711 | HDA2208 | 4335765 | Os04g33480 |
| Class II | | HDA704 | HDA2209 | 3037679 | Os07g06980 |
| | | HDA713 | HDA2213 | 3042941 4343829 | Os07g41090 |
| ClassIII | | HDA706 | HDA2210 | 4341352 | Os06g37420 |
| Unclassified | | HDA712 | HDA2211 | 4338920 | Os05g36920 |
| | HDAC10 | HDA714 | HDA2214 | 4351682 | Os12g08220 |
| | | HDA716 | HDA2212 | 4338921 | Os05g36930 |

Complex protein (Sin3 homologs)

| | | | | | |
|---|---|---|---|---|---|
| SNT | | SNT701 | SNT2201 | 3031797 3159524 | Os01g01960 |
| | | | | 4326133 | |
| | | SNT702 | SNT2202 | 3036512 4337520 | Os05g01020 |
| SIR2 | SRT1 | SRT701(HDA708) | SRT2201 | 3034298 4335342 | Os04g20270 |
| SRT | SRT2 | SRT702 | SRT2202 | 4351669 | Os12g07950 |

The expression profiles of histone acetylase and deacetylase genes in the whole growth period of rice were analyzed using the chip database of our laboratory (http://crep.ncpgr.cn/) (Wang et al., A dynamic gene expression atlas covering the entire life cycle of rice. Plant J, 2009). This was done by inputting gene accession numbers in the database to conduct searches to obtain chip signal values. Ten common tissues were selected from varieties “Minghui 63”, “Zhenshan 97” and “Shanyou 63”, i.e. endosperm, callus, leaf, sheath, plumule, flag leaf, stem, panicle at stage V, panicle at stage III, and root, and the signal values for these tissues were converted to log2 values. For each of the genes, the signal values for those various tissues were averaged, and then the differences between each of the signal values and the average value were calculated.

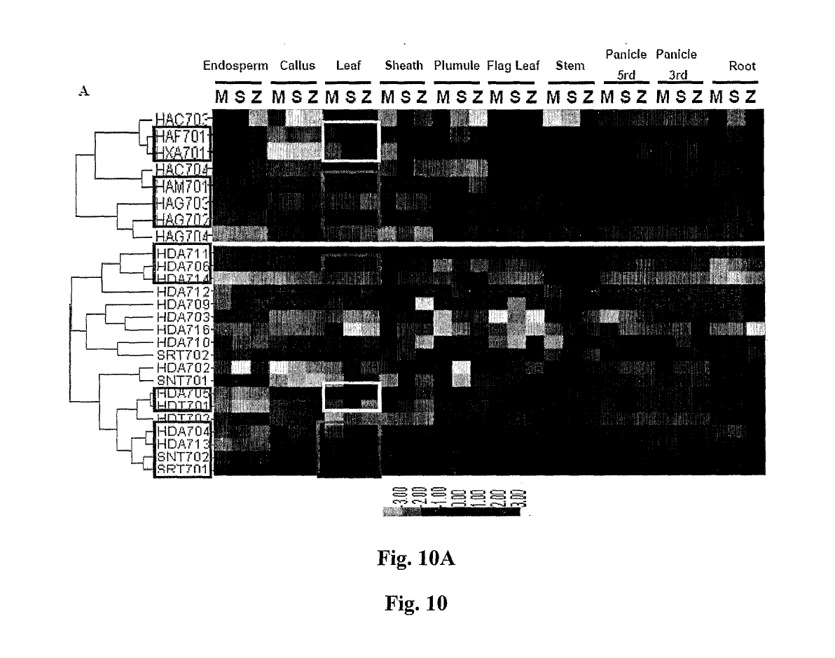

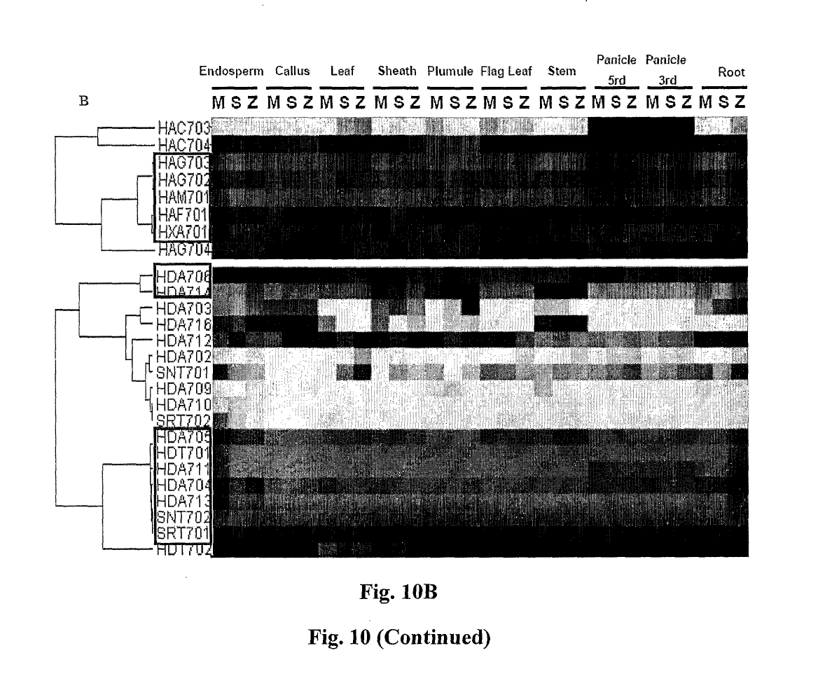

The differences obtained were subjected to cluster analysis using Cluster software and to phylogenetic analysis using Treeview software. The results obtained are shown in FIG. 10A.

In order to obtain a better analysis of the results of chip clustering, using the above method, for each of the various tissues, the relative signal values of various genes in the same tissue were calculated and subjected to cluster analysis. The results obtained are shown in FIG. 10B.

Genes having relatively high expression in the various tissues (HAF701, HXA701, HAM701, HAG702, HAG703, HDA706, HDA711, HDA714, HDA705, HDT701, HDA704, HDA713, SNT702 and SRT701) were selected for further analysis.

As the key tissue that influences flowering time is leaf, the levels of expression of these genes in the leaves of those three rice varieties were analyzed, leading to the finding that these genes could be classified into three groups. The first group includes HAM701, HAG702 and HAG703, whose expressions were relatively high in “Minghui 63” and relatively low in “Zhenshan 97”. The second group includes HAF701, HXA701, HDA706, HDA714, HDA704, HDA713, SNT702 and SRT701, whose expressions were relatively low in “Minghui 63” and relatively high in “Zhenshan 97”. The three group includes HDT701 and HDA705, which had a similar expression in “Minghui 63” and “Zhenshan 97” and a relatively high expression in “Shanyou 97”.

In summary, most acetylase genes had a relatively high level of expression in “Minghui 63” but a relatively low level in “Zhenshan 97”, while most deacetylase genes had a relatively low level of expression in “Minghui 63” but a relatively high level in “Zhenshan 97”. This would result in a relatively high degree of acetylation in “Minghui 63” as compared to “Zhenshan 97”, which had a very low acetylation level. The results of the analysis were in agreement with the results of Western blotting in Example 7.

1-9. (canceled)

10. A transformed plant exhibiting an improved property as compared to a control plant, wherein said improved property is selected from the group consisting of enhanced water use efficiency, increased yield, enhanced nitrogen use efficiency, altered flowering time, and altered seed setting, or combinations thereof, and wherein said plant expresses a polypeptide having a sequence with at least about 70%, 75%, 80%, 85%, 90%, or 95% identity to SEQ ID NO:2.

11. The transformed plant according to claim 10, wherein said plant is a crop plant.

12. A seed meal obtained from a seed of the transformed plant according to claim 10.

13. A transgenic plant cell comprising a recombinant DNA construct, said construct comprising a promoter functional in a plant cell operably linked to a polynucleotide that, when expressed in said plant cell, encodes a polypeptide having a sequence with at least about 70%, 75%, 80%, 85%, 90%, or 95% identity to SEQ ID NO:2, wherein said plant cell is selected by screening a population of transgenic plant cells that have been transformed with said construct for altered histone acetylation.

14. The transgenic plant cell of claim 13, wherein said plant cell is part of a transgenic plant.

15. The transgenic plant cell of claim 14, wherein said plant cell is part of a corn, soybean, cotton, canola, alfalfa, wheat, rice, sugarcane, or sugar beet plant.

16. The transformed plant according to claim 11, wherein said crop plant is a corn, soybean, cotton, canola, alfalfa, wheat, rice, sugarcane, or sugar beet plant.

17. A method of producing a transformed plant with an improved property as compared to a control plant, said method comprising:
(a) transforming plant cells with a recombinant DNA construct, said construct comprising a promoter functional in a plant cell operably linked to a polynucleotide encoding a polypeptide having a sequence with at least about 70%, 75%, 80%, 85%, 90%, or 95% identity to SEQ ID NO:2; and
(b) regenerating a plant from the transformed plant cells,
wherein said improved property is selected from the group consisting of enhanced water use efficiency, increased yield, enhanced nitrogen use efficiency, altered flowering time, and altered seed setting, or combinations thereof.


SEQUENCE LISTING (14 sequences)

SEQ ID NO: 1 - DNA, 894 bp, Oryza sativa
  gene (1)..(894)
  CDS (1)..(894)
atggagttctggggtcttgaagtcaagcctggacagactgtcaaatgtgagcctgaagatgaacgctttttgcacctttctcaggctgctcttggggaatcaaagaaaggatctgacaatgcagtaatgtatgttaaaactgatgatcaaaagctagtcattggaaccctctcagctgacaagttccctcaaatccagtttgatttggtctttgacaaagagtttgagctgtcacacacttcaaagactgctagtgtgttcttttctggctacaaagtttcccagccggctgaggaagatgaaatggattttgattctgaagaagttgaagatgaagaggaggaagaaaagatcattccagctcccagggcaaatggcaaagttgaagggaaggaaaatgagcagaaaaaacaaggcaagacagattcttcagcttcaaaatcaaaggctgcagtgaatgacgatgatgatgatgatgacagtgcagaggatgattctcaggacgaagatctttctcctgaggatgatgatgatgattcttctgaggatgattccagcgaagatgatgaggatgagagtgacgaggaagatactcccaagaagccagagactggaaagaggaaagtagctgaaattgtgttgaagacaccttcgtctgataagaaagcaaagattgctacaccgtcaggccagaagacaggtgacaagaagggtgtccatgtagcaactccacatccggcaaagcaggctagcaagacccccgtgaatgacaagtcaaaggagaagtccccaaaatccggtggtgggtcaatttcttgcaagtcatgcagcaagacgttcaacagtgaaatggctctgcaatctcactcgaaggccaagcaccccgccaagtga

SEQ ID NO: 2 - protein, 297 aa, Oryza sativa
MEFWGLEVKPGQTVKCEPEDERFLHLSQAALGESKKGSDNAVMYVKTDDQKLVIGTLSADKFPQIQFDLVFDKEFELSHTSKTASVFFSGYKVSQPAEEDEMDFDSEEVEDEEEEEKIIPAPRANGKVEGKENEQKKQGKTDSSASKSKAAVNDDDDDDDSAEDDSQDEDLSPEDDDDDSSEDDSSEDDEDESDEEDTPKKPETGKRKVAEIVLKTPSSDKKAKIATPSGQKTGDKKGVHVATPHPAKQASKTPVNDKSKEKSPKSGGGSISCKSCSKTFNSEMALQSHSKAKHPAK

SEQ ID NO: 3 - DNA, 29 bp, artificial
  synthetic
gggggtaccccgattccgatggagttctg

SEQ ID NO: 4 - DNA, 30 bp, artificial
  synthetic
gggggatccgaagctcagttcgcaccacag

SEQ ID NO: 5 - DNA, 35 bp, artificial
  synthetic
gggactagtggtaccggctgcagtgaatgacgatg

SEQ ID NO: 6 - DNA, 35 bp, artificial
  synthetic
ggggagctcggatcctcacttggcggggtgcttgg

SEQ ID NO: 7 - DNA, 29 bp, artificial
  synthetic
ggggaattcccgattccgatggagttctg

SEQ ID NO: 8 - DNA, 29 bp, artificial
  synthetic
gggggatcccttggcggggtgcttggcct

SEQ ID NO: 9 - DNA, 21 bp, artificial
  synthetic
cgctttttgcacctttctcag

SEQ ID NO: 10 - DNA, 19 bp, artificial
  synthetic
actttgccatttgccctgg

SEQ ID NO: 11 - DNA, 21 bp, artificial
  synthetic
agaagaagatgttggcgacct

SEQ ID NO: 12 - DNA, 20 bp, artificial
  synthetic
gtcctgcgggtaaatagctg

SEQ ID NO: 13 - DNA, 20 bp, artificial
  synthetic
tatggtcaaggctgggttcg

SEQ ID NO: 14 - DNA, 20 bp, artificial
  synthetic
ccatgctcgatggggtactt